(19) United States
(12) Patent Application Publication
(10) Pub. No.: US 2019/0150848 A1
(43) Pub. Date: May 23, 2019

(54) IMAGE PROCESSING APPARATUS, OPERATION METHOD PERFORMED BY IMAGE PROCESSING APPARATUS AND RECORDING MEDIUM

(71) Applicant: OLYMPUS CORPORATION, Tokyo (JP)

(72) Inventors: Takehito HAYAMI, Tokyo (JP); Yamato KANDA, Tokyo (JP); Takashi KONO, Tokyo (JP)

(73) Assignee: OLYMPUS CORPORATION, Tokyo (JP)

(21) Appl. No.: 16/237,838

(22) Filed: Jan. 2, 2019

Related U.S. Application Data
(63) Continuation of application No. PCT/JP2016/070745, filed on Jul. 13, 2016.

(51) Int. Cl. A61B 5/00; G06T 7/44; G06T 7/571; G06T 7/00

(52) U.S. Cl. CPC A61B 5/7264; G06T 7/44; G06T 7/571; G06T 2207/10024; A61B 5/4887; G06T 2207/30096; G06T 2207/10068; G06T 7/0016

(57) ABSTRACT

An image processing apparatus includes: a processor comprising hardware, the processer being configured to execute: setting, in an image, an area of interest where classification is evaluated; calculating surface layer structure information representing a surface layer structure in the area of interest; calculating at least focus degrees of the outside of the area of interest in the image; and classifying the image based on the surface layer structure information and the focus degrees of the outside of the area of interest.

### CROSS-REFERENCE TO RELATED APPLICATION

This application is a continuation of International Application No. PCT/JP2016/070745, filed on Jul. 13, 2016, the entire contents of which are incorporated herein by reference.

### BACKGROUND

The present disclosure, for example, relates to an image processing apparatus that classifies a group of intraluminal images that are acquired by capturing images of the inside of the lumen of a living body, an operation method performed by an image processing apparatus, and an operation program for an image processing apparatus.

A technology to calculate focus degrees of multiple locations in an image of one frame has been known. For example, according to Japanese Laid-open Patent Publication No. 2009-258284, a first high-frequency intensity that is lost when a blur or camera shake occurs and a second high-frequency intensity whose value is relatively larger than that of the first high-frequency intensity even when a blur or camera shake occurs and that contains frequency components on a lower-band side are extracted from an image and furthermore a noise parameter is set by calculating an average noise amplitude in the image. According to Japanese Laid-open Patent Publication No. 2009-258284, by calculating a ratio of the first high-frequency intensity to a sum of the second high-frequency intensity and the noise parameter, the focus degrees in multiple positions in the image are calculated.

### SUMMARY

An image processing apparatus according to one aspect of the present disclosure includes: a processor comprising hardware, the processer being configured to execute: setting, in an image, an area of interest where classification is evaluated; calculating surface layer structure information representing a surface layer structure in the area of interest; calculating at least focus degrees of the outside of the area of interest in the image; and classifying the image based on the surface layer structure information and the focus degrees of the outside of the area of interest.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a functional configuration of an image processing apparatus according to a first embodiment;

FIG. 2 is a flowchart illustrating image processing that is performed by the image processing apparatus according to the first embodiment; FIG. 3 is a flowchart illustrating a process of calculating focus degrees of the outside of an area of interest that is executed by an area-of-interest-outside degree-of-focus calculator;

FIG. 4 is a block diagram illustrating a functional configuration of an image processing apparatus according to Modification 1 of the first embodiment;

FIG. 5 is a flowchart illustrating image processing that is performed by the image processing apparatus according to Modification 1 of the first embodiment;

FIG. 6 is a flowchart illustrating a process of calculating focus degrees of the outside of an area of interest that is executed by an area-of-interest-outside degree-of-focus calculator;

FIG. 7 is a flowchart illustrating image processing performed by an image processing apparatus according to Modification 2 of the first embodiment;

FIG. 8 is a block diagram illustrating a functional configuration of an image processing apparatus according to a second embodiment;

FIG. 9 is a flowchart illustrating image processing that is performed by the image processing apparatus according to the second embodiment;

FIG. 10 is a flowchart illustrating a process of calculating focus degrees of the outside of an area of interest that is executed by an area-of-interest-outside degree-of-focus calculator;

FIG. 11 is a diagram illustrating setting of a reference area that is performed by a reference area setting unit;

FIG. 12 is a block diagram illustrating a functional configuration of an image processing apparatus according to a third embodiment;

FIG. 13 is a flowchart illustrating image processing that is performed by the image processing apparatus according to the third embodiment;

FIG. 14 is a flowchart illustrating a process of classifying an intraluminal image that is executed by an image classifier;

FIG. 15 is a flowchart illustrating image processing that is performed by an image processing apparatus according to a fourth embodiment; and

FIG. 16 is a flowchart illustrating a process of calculating focus degrees of the outside of an area of interest that is executed by an area-of-interest-outside degree-of-focus calculator.

### DETAILED DESCRIPTION

The present embodiment represents an image processing apparatus that classifies intraluminal images that are captured by an endoscope. An intraluminal image is a color image having pixel levels (pixel values) corresponding to color components of R (red), G (green) and B (blue) in respective pixel positions.

### First Embodiment

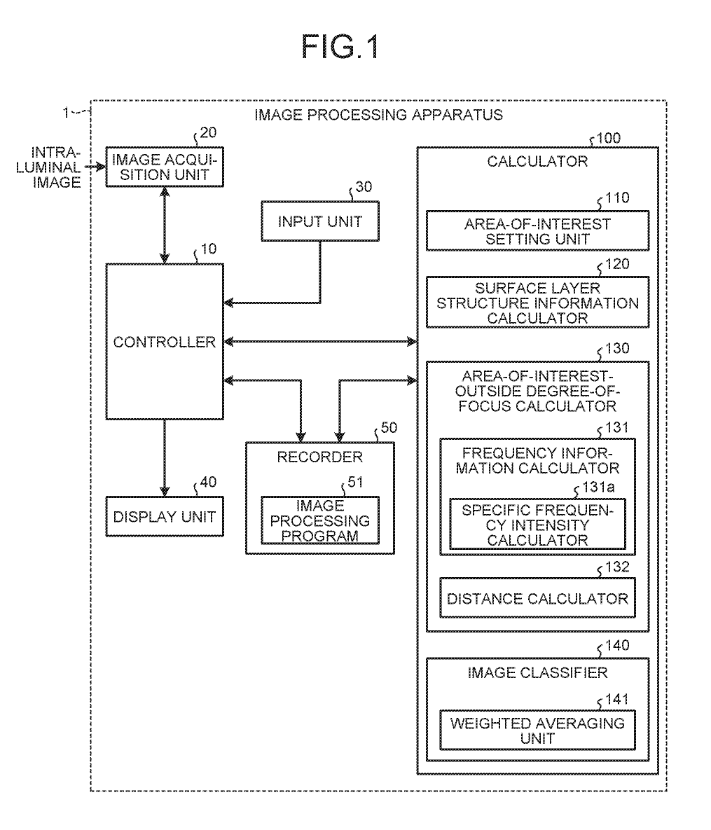

FIG. 1 is a block diagram illustrating a functional configuration of an image processing apparatus according to a first embodiment of the present disclosure. An image processing apparatus 1 according to a first embodiment classifies an intraluminal image based on a group of intraluminal images, an area of interest, such as a lesion area that is suspected as being a neoplastic lesion, and information on the outside of the area of interest.

The image processing apparatus 1 includes a controller 10 that controls entire operations of the image processing apparatus 1; an image acquisition unit 20 that acquires a group of intraluminal images that are generated by an imaging device by capturing images of the inside of the lumen; an input unit 30 that inputs signals corresponding to external operations to the controller 10; a display unit 40 that displays various types of information and images; a recorder 50 that stores image data that is acquired by the image acquisition unit 20 and various programs; and a calculator 100 that executes given image processing on the image data.

The controller 10 is realized by hardware, such as a central processing unit (CPU). The controller 10 reads the various programs that are recorded in the recorder 50 and thus, according to the group of intraluminal images that is input from the image acquisition unit 20 and signals that are input from the input unit 30, etc., performs transfer of instructions and data to the components of the image processing apparatus 1, etc., and perform overall control on entire operations of the image processing apparatus 1.

The image acquisition unit 20 is configured properly according to the mode of the system including a medical imaging device. For example, when the imaging device is connected to the image processing apparatus 1, the image acquisition unit 20 consists of an interface that loads the group of intraluminal images that are generated by the imaging device. When a server that saves the group of intraluminal images that are generated by the imaging device is set, the image acquisition unit 20 consists of a communication device that is connected to a server, etc., and performs data communication with the server to acquire the group of intraluminal images. Alternatively, the group of intraluminal images that are generated by the imaging device may be delivered with a portable recording medium. In this case, the image acquisition unit 20 consists of a reader device to which the portable recording medium is detachably attached and that reads the recorded group of intraluminal images.

The input unit 30 is, for example, realized by an input device including a keyboard, a mouse, a touch panel and various switches. The input unit 30 outputs input signals that are generated according to external operations on the input devices to the controller 10.

The display unit 40 is realized by a display device, such as a liquid crystal display (LCD) or an electroluminescence (EL) display. The display unit 40 displays various screens containing the group of intraluminal images under the control of the controller 10.

The recorder 50 is realized by an information storage device including various IC memories, such as a ROM or a RAM that is an updatable and recordable flash memory, a hard disk that is incorporated or connected by a data communication terminal or a CD-ROM, and a device that reads the information storage device. The recorder 50 stores, in addition to the group of intraluminal images that is acquired by the image acquisition unit 20, a program for causing the image processing apparatus 1 to operate and causing the image processing apparatus 1 to execute various functions, data that is used during execution of the program, etc. Specifically, the recorder 50 stores an image processing program 51 to classify the group of intraluminal images, a threshold that is used in the image processing, the result of classification performed by the calculator 100, etc.

The calculator 100 is realized by hardware, such as a CPU. The calculator 100 reads the image processing program 51 to execute the image processing to classify a group of intraluminal images.

The calculator 100 includes an area-of-interest setting unit 110 that sets, in an acquired image, an area of interest where image classification is evaluated; a surface layer structure information calculator 120 that calculates information representing a surface layer structure in the area of interest; an area-of-interest-outside degree-of-focus calculator 130 that calculates focus degrees of the outside of the area of interest; and an image classifier 140 that classifies the image based on the surface layer structure information and the focus degrees of the outside of the area of interest.

The area-of-interest-outside degree-of-focus calculator 130 includes a frequency information calculator 131 that calculates frequency information on an intraluminal image and a distance calculator 132. Furthermore, the frequency information calculator 131 includes a specific frequency intensity calculator 131a that calculates an intensity of a specific frequency band of an image.

The image classifier 140 includes a weighted averaging unit 141 that performs weighted averaging on the focus degrees of the outside of the area of interest depending on distances that are calculated by the distance calculator 132 to calculate a focus degree of the area of interest.

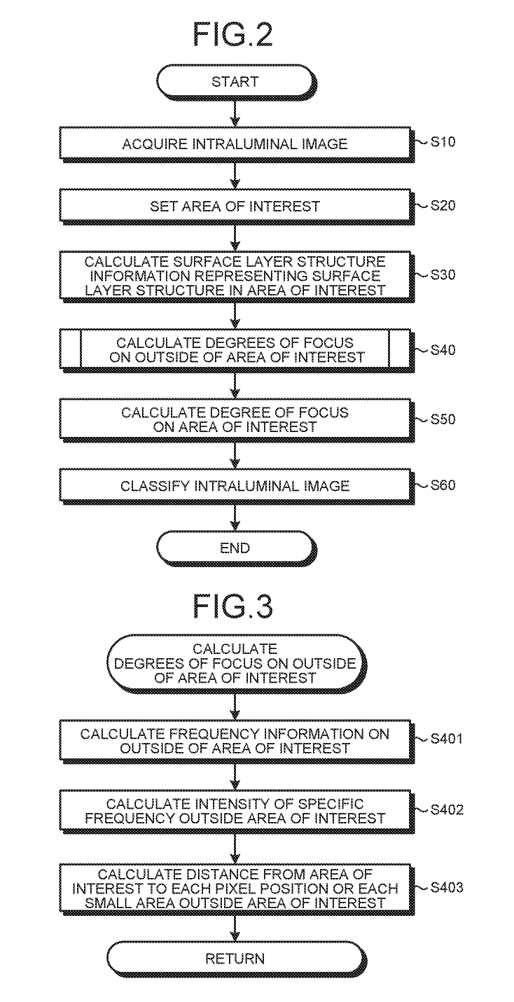

Operations of the image processing apparatus 1 will be described. FIG. 2 is a flowchart illustrating image processing that is performed by the image processing apparatus according to the first embodiment of the present disclosure. First of all, at step S10, the image processing apparatus 1 acquires an intraluminal image via the image acquisition unit 20. In the first embodiment, an intraluminal image that is generated by applying illumination light (white light) containing wavelength components of R, G and B to the inside of the lumen with an endoscope to capture an image and that has pixel values (R values, G values and B values) corresponding to the wavelength components in pixel positions, respectively. The illumination light is not limited to the aforementioned white light, and the illumination light may be special light containing narrowband wavelength components of G and B or illumination light containing a single narrowband light of at least one of R, G and B. For example, an intraluminal image that is generated by applying special light containing narrowband wavelength components of G and B to the inside of the lumen to capture an image and that has pixel values (G values and B values) corresponding to the wavelength components in pixel positions, respectively.

At step S20, the calculator 100 sets an area of interest. Specifically, the area-of-interest setting unit 110 detects a location of interest in the intraluminal image and sets an area of interest containing the location of interest. The area-of-interest setting unit 110 sets an area of interest by an input made by the user or by using a known method, such as known snake (reference literature: CG-ARTS Association “Digital Image Processing” revised new version, pp, 210) or graph cut (reference literature: CG-ARTS Association “Digital Image Processing”, revised new version, pp, 212). Alternatively, by performing the possible polyp detection process described in Japanese Laid-open Patent Publication No. 2007-244518, an area of polyp may be extracted as an area of interest. Alternatively, an area of interest may be detected by DPM ((deformable parts model), reference literature: “A Discriminatively trained, Multiscale, Deformable Part Model”, Pedro Felzenszalb, University of Chicago), machine learning using deep learning enabling detection of an area without designing a characteristic amount (reference literature: “Learning deep architectures for AI”, Y. Bengio), or the like. Furthermore, instead of polyp, a lesion, such as tumor, or an abnormal part may be detected and an area of interest containing any one of them may be set.

At the following step S30, the calculator 100 calculates surface layer structure information that represents a surface layer structure in the area of interest. Specifically, the surface layer structure information calculator 120 calculates information representing a surface layer structure in the area of interest that is set. The information representing a surface layer structure that is calculated herein is, for example, an edge strength that is calculated by applying known edge extraction processing (reference literature: CG-ARTS Association “Digital Image Processing”, revised new version, pp, 105). When multiple sets of information are obtained, for example, when edge strengths are obtained with respect to respective pixel positions, the surface layer structure information calculator 120 uses a representative value, such as the average or the mode, as the surface layer structure information. The surface layer structure information calculator 120 may calculate frequency information on the area of interest as the surface layer structure information.

At the following step S40, the calculator 100 calculates focus degrees of the outside of the area of interest. FIG. 3 is a flowchart illustrating a process of calculating the focus degrees of outside of an area of interest that is executed by the area-of-interest-outside degree-of-focus calculator.

At step S401, the frequency information calculator 131 calculates frequency information on the outside of the area of interest. Specifically, multiple pixels of the imaging device are arrayed in a matrix and the frequency information calculator 131 calculates frequency information F(u,v) on an image I(x,y) by Equation (1) below where (x,y) denotes a set of coordinates of a pixel. In an image consisting of wavelength components of colors of R, G and B, G components and B components are close to a blood absorption band where a subject (blood vessels) representing a contrast change tends to be seen and saturation is less likely to occur. For this reason, the frequency information calculator 131 calculates frequency information F(u,v) based on G components and B components. Before the process step S401, the calculator 100 may remove saturation and halation caused by an optical system or an illumination system that can cause low accuracy.

$$F ( u , v ) = \frac{1}{N} {\sum }_{x = 0}^{N - 1} {\sum }_{y = 0}^{N - 1} I ( x , y ) exp \{ - \frac{j 2 π ( ux + vy )}{N} \} , \qquad ( 1 )$$

where j is an imaginary unit, j=√(−1),
u is a spatial frequency in an x direction, and
v is a spatial frequency in a y direction.

At step S402 following step S401, the specific frequency intensity calculator 131a extracts a frequency w=√(u²+v²) in a specific range in the obtained frequency information F(u,v) and cuts other frequencies off, thereby calculating an intensity of the specific frequency outside the area of interest. The specific range herein covers characteristic frequencies representing a surface layer structure, for example, characteristic frequencies representing texture, such as the thickness of a blood vessel, and the specific range is set previously. Furthermore, frequency information F′(u,v) that is extracted is converted by Equation (2) below into a post-process image I′(x,y).

$${I}^{′} ( x , y ) = \frac{1}{N} {\sum }_{u = 0}^{{N}^{′} - 1} {\sum }_{v = 0}^{{N}^{′} - 1} {F}^{′} ( u , v ) exp \{ \frac{j 2 π ( ux + vy )}{{N}^{′}} \} \qquad ( 2 )$$

The specific frequency intensity calculator 131a takes, as a focus degree in each pixel position, an absolute value |I′ (x,y)| representing the intensity of the specific frequency band. The focus degree of corresponds to the intensity of the specific frequency. The specific frequency intensity calculator 131a may set a small area containing multiple pixel positions and a representative value of the focus degrees in the small area may be calculated as the focus degree of the small area. The representative value may be, for example, a mean, a median, a mode, a maximum or a minimum.

In another method of calculating an intensity of a specific frequency, the specific frequency intensity calculator 131a sets, in the intraluminal image, a small area whose longitudinal and lateral lengths are equal to each other, uses the small area as I(x,y) to calculate frequency information F(u,v) for each small area by Equation (1), and calculates a power spectrum by Equation (3) below:

p(u, v)=|F (u, v)|² (3)

The specific frequency intensity calculator 131a extracts a frequency w=√(u²+v²) in a specific range in the power spectrum p(u,v) and cuts other frequencies off, thereby calculating an intensity of the specific frequency. The specific frequency intensity calculator 131a calculates a representative value of the extracted power spectrum p(u,v) as a focus degree.

At step S403 following step S402, the distance calculator 132 calculates a distance from the area of interest to each pixel position or each small area outside the area of interest. The distance that is calculated by the distance calculator 132 is a distance from the set of coordinates of the area of interest to a set of coordinates outside the area of interest on the image or a difference between a shooting distance to a subject reflected in the area of interest and a shooting distance to the subject reflected in the outside of the area of interest.

When calculating a distance on the image, the distance calculator 132 calculates a center of gravity of the area of interest and calculates a distance from the set of coordinates of the pixel position in which the center of gravity is positioned to a set of coordinates of each pixel position outside the area of interest.

When calculating a difference between shooting distances, first of all, the distance calculator 132 determines a representative value (for example, a mean, a median, a mode, a maximum or a minimum) of shooting distances to the subject reflected in the area of interest. The distance calculator 132 then calculates a difference between a shooting distance to each pixel position outside the area of interest and the representative value of shooting distances that is determined by the area of interest. The shooting distance to the subject on the intraluminal image can be calculated by a known calculation method. For example, pixel values of wavelength components, which will be described below, may be used to calculate the shooting distance, a stereo image may be acquired to calculate the distance or the distance may be calculated based on a result of measuring a distance using a distance measurement sensor.

After the distance calculator 132 calculates the distances, the operation of the calculator 100 returns to the main routine. Based on the calculated distances, the area outside the area of interest where a focus degree is calculated may be limited to one whose corresponding distance is within a given range. In this case, the distance calculator 132 calculates distances before the frequency information calculator 131 calculates frequency information.

At step S50 following step S40, the image classifier 140 calculates a focus degree of the area of interest. Specifically, the weighted averaging unit 141 calculates a focus degree of the area of interest by performing weighted averaging on the focus degrees of the outside of the area of interest depending on the distances. The weighted averaging unit 141 calculates a focus degree fₜ of the area of interest by Equation (4) below:

$${f}_{t} = \frac{1}{{\sum }_{i = 1}^{K} {w}_{i}} {\sum }_{i = 1}^{K} {w}_{i} {f}_{i} \qquad ( 4 )$$

where K is the number of pixels or the number of areas outside the area of interest,
wᵢ is a weight corresponding to the distance, and
fᵢ is a focus degree of the outside of the area of interest. The weight wᵢ increases as the distance reduces and reduces as the distance increases. The weight wᵢ is, for example, calculated by Equation (5) below:

$${w}_{i} = k exp ( - \frac{{d}_{i}^{2}}{2 {σ}^{2}} ) \qquad ( 5 )$$

where k is a given coefficient,
σ is a standard deviation, and
dᵢ is a distance between the area of interest and the outside of the area of interest. The equation is not limited to Equation (5) as long as the equation enables calculation of a weight wᵢ that increases as the distance reduces and that reduces as the distance increases.

At step S60 following step S50, the image classifier 140 classifies the intraluminal image based on the focus degree fₜ on the area of interest that is calculated by the weighted averaging unit 141 and the surface layer structure information that is calculated at step S30. Specifically, the image classifier 140 classifies the intraluminal image as a focused image having a surface layer structure in the area of interest, a focused image having no surface layer structure or an unfocused image that is not focused. The image classifier 140 classifies an intraluminal image as a focused image having a surface layer structure when the information representing a surface layer structure is equal to or larger than a pre-set value. The image classifier 140 classifies an intraluminal image as a focused image having no surface layer structure when the information representing a surface layer structure is smaller than the pre-set value and the focus degree fₜ on the area of interest is equal to or larger than a pre-set value. The image classifier 140 classifies an intraluminal image as an unfocused image having no surface layer structure when both the information representing a surface layer structure and the focus degree fₜ on the area of interest are smaller than the pre-set values. The value that is previously set for the information representing a surface layer structure is a value that is set with respect to the intensity of the specific frequency and is a value based on which it can be determined that the intraluminal image has a surface layer structure and the surface layer structure is seen clearly in the intraluminal image. The value that is previously set for the focus degree fₜ on the area of interest is a value that is set for the focus degree fₜ and is a value based on which it can be determined that a subject in the area of interest is seen clearly. The controller 10 then records the result of classification in the recorder 50 in association with the intraluminal image and displays the result on the display unit 40. The controller 10 repeats the above-described classification process at the timing of acquisition of an intraluminal image by the image acquisition unit or timing of satisfaction of a condition that is set, or timing of each frame or each few frames.

As described above, according to the first embodiment of the present disclosure, an intraluminal image is classified based on surface layer structure information on an area of interest and a focus degree on the area of interest that is calculated according to the focus degree of the outside of the area of interest and distances from positions outside the area of interest to the area of interest and thus detailed classification of a group of intraluminal images is enabled. With a known method, a focus degree is not necessarily determined accurately due to absence of a subject representing contrast changes from an area of interest; however, according to the first embodiment, a focus degree of an area of interest is calculated by performing weight averaging on the focus degrees of the outside of the area of interest according to distances from the area of interest to determine whether the area of interest is focused and this enables accurate classification of the intraluminal image as a focused image containing existence and absence of a surface layer structure or as an unfocused image.

### Modification 1 of First Embodiment

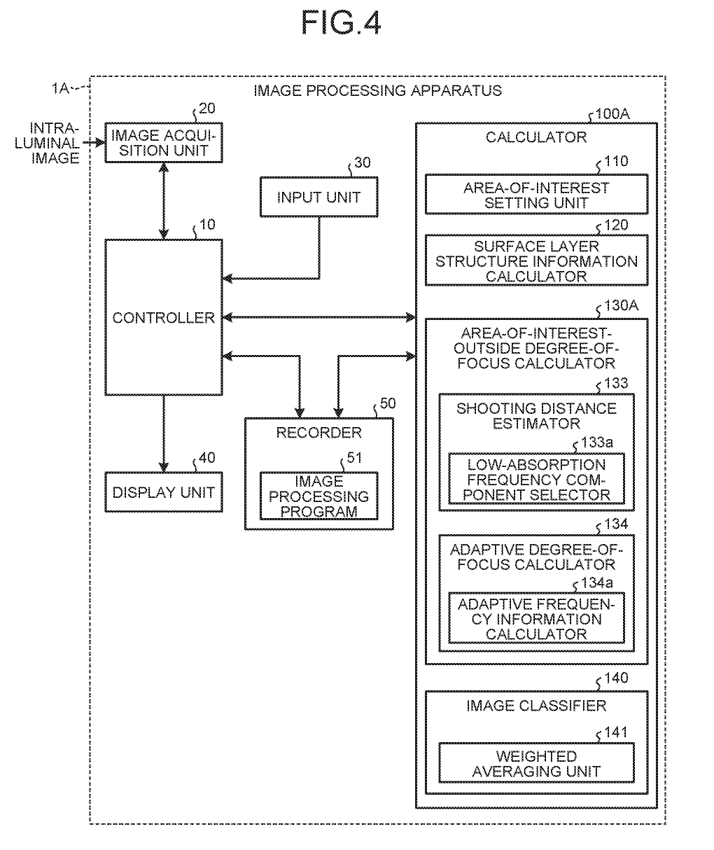

FIG. 4 is a block diagram illustrating a functional configuration of an image processing apparatus according to Modification 1 of the first embodiment of the present disclosure. The same components as those of the image processing apparatus 1 according to the first embodiment will be denoted with the same reference numbers as those of the first embodiment and described. An image processing apparatus 1A illustrated in FIG. 4 includes the controller 10 that controls entire operations of the image processing apparatus 1A; the image acquisition unit 20 that acquires image data that is generated by an imaging device by capturing images of the inside of the lumen; the input unit 30 that inputs signals corresponding to external operations to the controller 10; the display unit 40 that displays various types of information and images; the recorder 50 that stores image data that is acquired by the image acquisition unit 20 and various programs; and a calculator 100A that executes given image processing on the image data.

The calculator 100A includes the area-of-interest setting unit 110 that sets, in an acquired image, an area of interest where image classification is evaluated; the surface layer structure information calculator 120 that calculates information representing a surface layer structure in the area of interest; an area-of-interest-outside degree-of-focus calculator 130A that calculates focus degrees of the outside of the area of interest; and the image classifier 140 that classifies the image based on the surface layer structure information and the focus degree of the outside of the area of interest.

The area-of-interest-outside degree-of-focus calculator 130A includes a shooting distance estimator 133 that estimates a shooting distance to each pixel in the image and an adaptive degree-of-focus calculator 134 that calculates a focus degree based on information on different frequency bands according to the shooting distances. Furthermore, the shooting distance estimator 133 includes a low absorption frequency component selector 133a that selects a low absorption wavelength component with the lowest degree of absorption into and scattering in the living body. The adaptive degree-of-focus calculator 134 includes an adaptive frequency information calculator 134a that calculates information on frequency bands that differ adaptively according to the shooting distances.

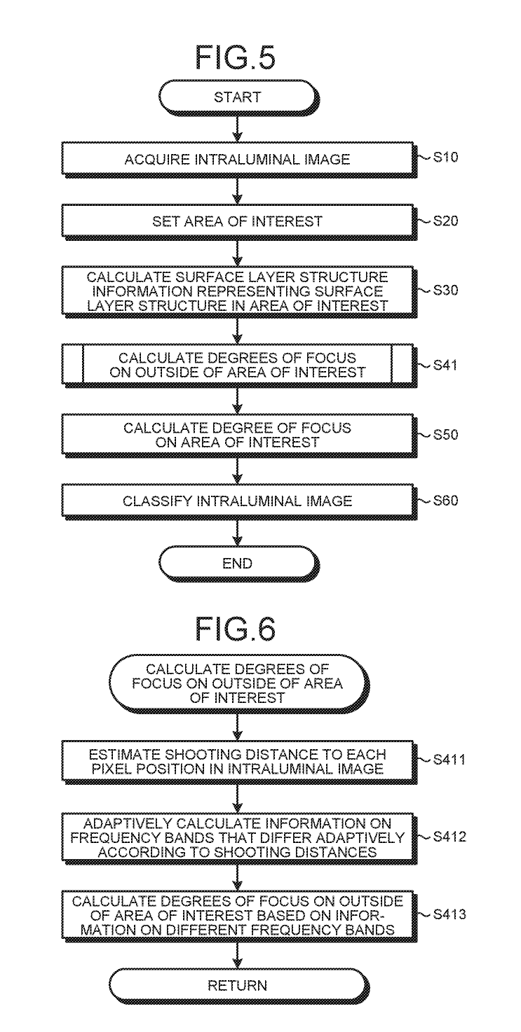

Operations of the image processing apparatus 1A will be described. FIG. 5 is a flowchart illustrating image processing that is performed by the image processing apparatus of Modification 1 of the first embodiment of the present disclosure. First of all, at step S10, the image processing apparatus 1A acquires an intraluminal image via the image acquisition unit 20.

At the following step S20, the calculator 100A sets an area of interest. As in the above-described first embodiment, the area-of-interest setting unit 110 detects a location of interest in the intraluminal image and sets an area of interest containing the location of interest.

At the following step S30, the calculator 100A calculates surface layer structure information representing a surface layer structure in the area of interest. As in the above-described first embodiment, the surface layer structure information calculator 120 calculates surface layer structure information representing a surface layer structure in the area of interest that is set.

At the following step S41, the calculator 100A calculates focus degrees of the outside of the area of interest. FIG. 6 is a flowchart illustrating a process of calculating focus degrees of the outside of the area of interest that is executed by the area-of-interest-outside degree-of-focus calculator.

At step S411, the shooting distance estimator 133 estimates a shooting distance to each pixel position in the image. There are various known methods of estimating a shooting distance. Modification 1 represents a method of estimating a shooting distance in which it is assumed that a subject to be imaged is a uniform diffuser based on the intraluminal image. Specifically, first of all, the low absorption frequency component selector 133a selects a low-absorption wavelength component with the lowest degree of absorption into or scattering in the living body. In Modification 1, the low absorption frequency component selector 133a will be described as one that selects R components to inhibit pixel values from lowering due to blood vessels that are seen on the mucous surface and to obtain pixel value information most correlated with the shooting distance to the mucous surface. In an image consisting of wavelength components of colors of R, G and B, R components are selected as R components are wavelengths apart from the blood absorption band and are long wavelength components and thus are less affected by scattering. Based on the pixel values of the low absorption wavelength components, the shooting distance estimator 133 estimates shooting distances to an uniform diffuser that is assumed. The shooting distance estimator 133 calculates an shooting distance that is estimated according to Equation (6) below:

$$r = \sqrt{\frac{I \times K \times cos θ}{L}} \qquad ( 6 )$$

where r is an shooting distance,
I is a radiant intensity of a light source;
K is a coefficient of diffuse reflection on the mucous surface,
θ is an angle formed by a normal vector on the mucous surface and a vector from the mucous surface to the light source, and

L is a pixel value of an R component of a pixel on which the mucous surface is reflected, for which an shooting distance is to be estimated. The radiant intensity I and the coefficient of diffuse reflection K are values that are previously set from values that are measured previously. The angle θ is a value that is determined by the positional relationship between the light source at the tip of the endoscope and the mucous surface and an average value is set previously.

Before executing step S411, the shooting distance estimator 133 may correct non-uniformity in pixel values due to the optical system and the illumination system that can cause accuracy of each process to lower and remove a non-mucosal area including specular reflection, residues and bubbles.

Modification 1 represents a method based on an image. Alternatively, shooting distances may be calculated based on a distance measurement sensor, etc. Alternatively, estimation of shooting distances need not necessarily be performed and pixel values that are correlated with shooting distances may be used to perform an adaptive process at the following stage.

At step S412, the adaptive frequency information calculator 134a calculates information on frequency bands that differ adaptively according to the shooting distances. The structure of the mucous surface that is reflected on the intraluminal image varies in size on the image according to an increase or decrease of the shooting distances and information on the frequency band also varies according to the shooting distances. Thus, when frequency information is calculated at step S401 in FIG. 3 described above, the range of the frequency w that is extracted from the frequency information F(u,v) is varied as a function of the shooting distance. The adaptive frequency information calculator 134a, for example, reduces the range of frequency w as the shooting distance increases.

At step S413, the adaptive degree-of-focus calculator 134 calculates focus degrees of the outside of the area of interest based on the information on different frequency bands that is calculated by the adaptive frequency information calculator 134a. The adaptive degree-of-focus calculator 134 calculates a focus degree from the frequency information, which is obtained at step S412, in the same manner as in the above-escribed first embodiment. For example, the adaptive degree-of-focus calculator 134 converts extracted frequency information F′(u,v) to a post-process image I′(x,y) by Equation (2) above.

The adaptive degree-of-focus calculator 134 takes, as a focus degree of each pixel position, an absolute value |I′(x,y)| in the post-process image I′(x,y). The adaptive degree-of-focus calculator 134 may set a small area and calculate a representative value of focus degrees in the small area as a focus degree of the small area. The representative value may be, for example, a mean, a median, a mode, a maximum or a minimum. Thereafter, the operation of the calculator 100 returns to the main routine.

At step S50 following step S41, the image classifier 140 calculates a focus degree of the area of interest. Specifically, the weighted averaging unit 141 calculates a focus degree of the area of interest by performing weighted averaging on the focus degrees of the outside of the area of interest depending on the distances. The weighted averaging unit 141 calculates a focus degree fₜ by Equation (4) above.

At step S60 following step S50, the image classifier 140 classifies the intraluminal image based on the focus degree fₜ on the area of interest that is calculated by the weighted averaging unit 141 and the surface layer structure information that is calculated at step S30. As described above, the image classifier 140 classifies the intraluminal image as a focused image having a surface layer structure in an area of interest, a focused image having a surface layer structure or an unfocused image that is not focused.

As described above, according to Modification 1 of the first embodiment of the present disclosure, an intraluminal image is classified based on the surface layer structure information on an area of interest and a focus degree of the area of interest based on focus degrees of the outside of the area of interest and information on frequency bands that are determined adaptively according to the shooting distances and thus detailed classification of a group of intraluminal images is enabled.

The method of estimating an shooting distance taken by the shooting distance estimator 133 according to Modification 1 may be used for calculation of distances performed by the distance calculator 132 according to the above-described first embodiment.

### Modification 2 of First Embodiment

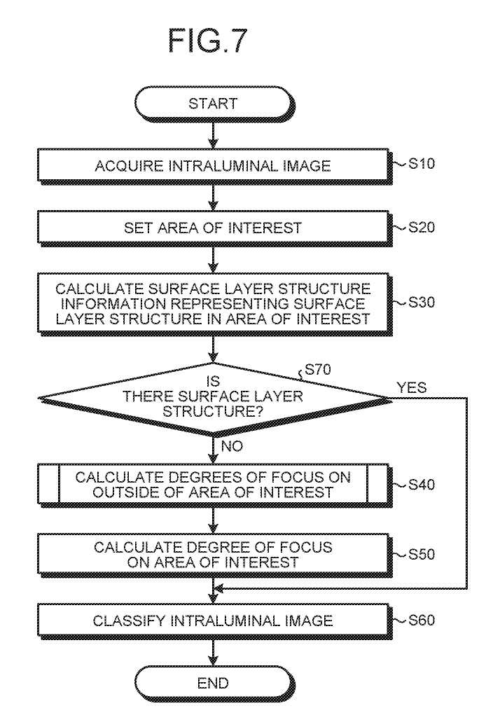

A configuration of an image processing apparatus according to Modification 2 of the first embodiment is the same as that of the image processing apparatus 1 according to the above-described first embodiment. FIG. 7 is a flowchart illustrating image processing that is performed by the image processing apparatus according to Modification 2 of the first embodiment. First of all, at step S10, the image processing apparatus 1 acquires an intraluminal image via the image acquisition unit 20.

At the following step S20, the calculator 100 sets an area of interest. As in the above-described first embodiment, the area-of-interest setting unit 110 detects a location of interest in the intraluminal image and sets an area of interest containing the location of interest.

At the following step S30, the calculator 100 calculates surface layer structure information representing a surface layer structure in the area of interest. As in the above-described first embodiment, the surface layer structure information calculator 120 calculates surface layer structure information on the area of interest that is set. Modification 2 will be described as one where an edge strength is calculated as the surface layer structure information.

At step S70 following step S30, the calculator 100 determines whether there is a surface layer structure from the result of calculation at step S30. The calculator 100 determines whether the surface layer structure information is equal to or larger than a pre-set value. As the surface layer structure information is an edge strength, the determination at step S70 corresponds to determination on whether the area of interest is focused in the intraluminal image. For this reason, as a setting value to be used herein, a value to determine whether the area of interest is focused from the surface layer structure information is set. When it is determined that there is a surface layer structure (YES at step S70), the calculator 100 moves to step S60. On the other hand, when it is determined that there is no surface layer structure (NO at step S70), the calculator 100 moves to step S40.

At the following step S40, the calculator 100 calculates focus degrees of the outside of the area of interest. The calculator 100 calculates focus degrees of the outside of the area of interest according to the flowchart illustrated in FIG. 3.

At step S50 following step S40, the image classifier 140 calculates a focus degree of the area of interest. Specifically, the weighted averaging unit 141 calculates a focus degree of the area of interest by performing weighted averaging on the focus degrees of the outside of the area of interest depending on the distances. The weighted averaging unit 141 calculates a focus degree fₜ by Equation (4) above.

At step S60 following step S50, the image classifier 140 classifies the intraluminal image based on at least the surface layer structure information. When it is determined that the area of interest has a surface layer structure (YES at step S70), the image classifier 140 classifies the intraluminal image as an image that has a surface layer structure and that is focused. On the other hand, when it is determined that the area of interest has no surface layer structure (NO at step S70), the image classifier 140 classifies the intraluminal image as a focused image that has no surface layer and as an unfocused image that has no surface layer structure and that is not focused based on the focus degree fₜ on the area of interest, which is calculated by the weighted averaging unit 141.

As described above, according to Modification 2 of the first embodiment of the present disclosure, an intraluminal image is classified based on surface layer structure information on an area of interest and a focus degree of the area of interest based on the focus degrees of the outside of the area of interest and information on frequency bands that are adaptively determined according to the shooting distances and thus detailed classification of a group of intraluminal images is enabled.

### Modification 2

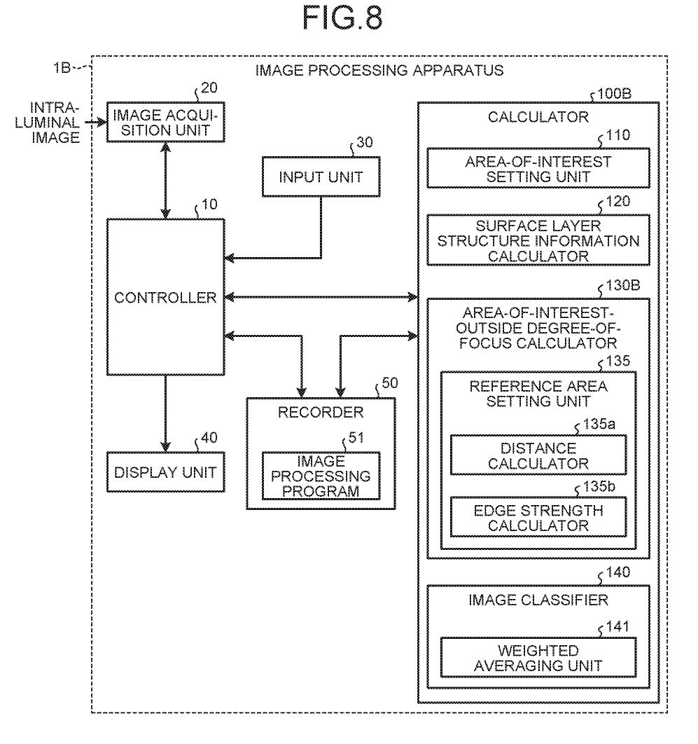

FIG. 8 is a block diagram illustrating a functional configuration of an image processing apparatus according to a second embodiment of the present disclosure. The same components as those of the image processing apparatus 1 according to the first embodiment, or the like, will be denoted with the same reference numbers as those of the first embodiment, or the like, and described. An image processing apparatus 1B illustrated in FIG. 8 includes the controller 10 that controls entire operations of the image processing apparatus 1B; the image acquisition unit 20 that acquires image data that is generated by capturing images of the inside of the lumen; the input unit 30 that inputs signals corresponding to external operations to the controller 10; the display unit 40 that displays various types of information and images; the recorder 50 that stores the image data that is acquired by the image acquisition unit 20 and various programs; and a calculator 100B that executes given image processing on the image data.

The calculator 100B includes the area-of-interest setting unit 110 that sets, in an acquired image, an area of interest where image classification is evaluated; the surface layer structure information calculator 120 that calculates information representing a surface layer structure in the area of interest; an area-of-interest-outside degree-of-focus calculator 130B that calculates focus degrees of the outside of the area of interest; and the image classifier 140 that classifies the image based on the surface layer structure information and the focuses on the outside of the area of interest.

The area-of-interest-outside degree-of-focus calculator 130B includes a reference area setting unit 135 that sets a reference area such that the reference area contains only pixels whose corresponding distances are within a given range and furthermore no edge exists between the area of interest and the reference area. Furthermore, the reference area setting unit 135 includes a distance calculator 135a that calculates a distance from the area of interest to each pixel position in an intraluminal image and an edge strength calculator 135b that calculates an edge strength in the intraluminal image.

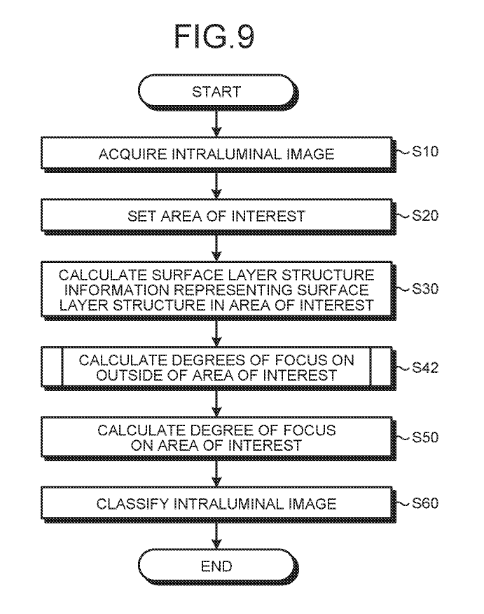

Operations of the image processing apparatus 1B will be described. FIG. 9 is a flowchart illustrating image processing that is performed by the image processing apparatus according to the second embodiment of the present disclosure. First of all, at step S10, the image processing apparatus 1 acquires an intraluminal image via the image acquisition unit 20.

At the following step S20, the calculator 100A sets an area of interest. As in the above-described first embodiment, the area-of-interest setting unit 110 detects a location of interest in the intraluminal image and sets an area of interest containing the location of interest.

At the following step S30, the calculator 100B calculates surface layer structure information representing a surface layer structure in the area of interest. As in the above-described first embodiment, the surface layer structure information calculator 120 calculates information representing a surface layer structure in the area of interest that is set.

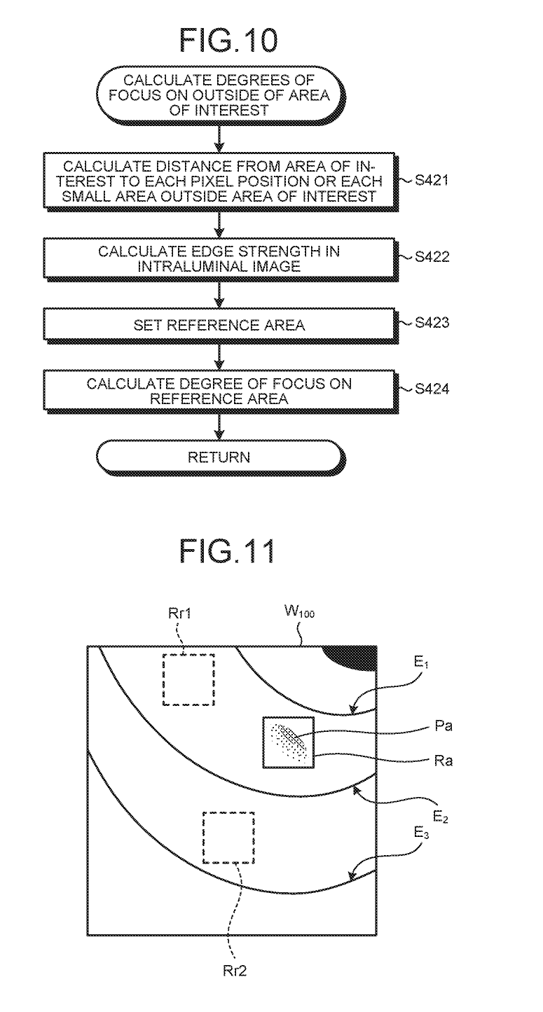

At the following step S42 the calculator 100B calculates focus degrees of the outside of the area of interest. FIG. 10 is a flowchart illustrating a process of calculating focus degrees of the outside of the area of interest that is executed by the area-of-interest-outside degree-of-focus calculator.

At step S421, the distance calculator 135a calculates a distance from the area of interest to each pixel position in the intraluminal image. The distance calculator 135a calculates distances using the same method as the calculation method performed by the distance calculator 132.

At step S422, the edge strength calculator 135b calculates an edge strength in the intraluminal image. By calculating an edge strength in the intraluminal image, the edge strength calculator 135b is able to detect an edge in the intraluminal image.

At step S423, the reference area setting unit 135 sets a reference area. The reference area setting unit 135 sets a reference area such that the reference area contains only pixels whose corresponding distances from the area of interest are within a pre-set given range and such that no edge having an intensity equal to or larger than a pre-set strength exists between the area of interest and the reference area. As a method of setting a reference area, for example, some thresholds are set for distances and the threshold process makes it possible to set a reference area with an interval that is set. The reference area setting unit 135 connects each pixel position and the position of center of gravity of the area of interest by a straight line. When the straight line intersects with an edge having a strength equal to or larger than the pre-set strength, the reference area setting unit 135 does not contain the pixel position in the reference area. Alternatively, it suffices if the reference area setting unit 135 determines not to set an area containing the pixel position as the reference area. Thus, at least one reference area is set between edges or in an area surrounded by an edge and the outer periphery of the image.

FIG. 11 is a diagram illustrating setting of a reference area that is performed by the reference area setting unit. According to FIG. 11, an intraluminal image W₁₀₀ contains edges E₁ to E₃ having strengths equal to or larger than the pre-set strength and a location of interest Pa and an area of interest Ra containing the area of location Ra is set. As illustrated in FIG. 11, as no edge having a strength equal to or larger than the pre-set strength exists between the area of interest Ra and a possible reference area Rr1, the possible reference area Rr1 can be set as a reference area. On the other hand, as an edge E₂ having a strength equal to or larger than the pre-set strength exists between the area of interest Ra and the possible reference area Rr2, the possible reference area Rr2 is not set as a reference area. Regardless whether the area of interest Ra contains an edge, when no edge exists between the area of interest Ra and a possible reference area, the reference area setting unit 135 sets the possible reference area as a reference area. As the method of setting a reference area, a method of setting a regular area, such as developing a rectangular area around grid points, or a method of setting a position or size of an area randomly are exemplified. Alternatively, a method of setting a reference area containing only pixels each of whose pixel degree is within a given degree, instead of distances, may be used to set a reference area, or whether to set a reference area may be determined based on the shooting distance or luminance. Note that FIG. 11 represents the area of interest and the reference area that are rectangular frames; however, the area of interest and the reference area are not limited thereto and the areas may be polygons other than quadrangles, ovals or circles or may have different sizes.

At step S424, the area-of-interest-outside degree-of-focus calculator 130B calculates a focus degree of each reference area. Specifically, the area-of-interest-outside degree-of-focus calculator 130B replaces the small area in the frequency information calculation at step S401 with the reference area and calculates focus degrees of the outside of the area of interest. Thereafter, the operation of the calculator 100 returns to the main routine.

At step S50 following step S42, the image classifier 140 calculates a focus degree of the area of interest. Specifically, the weighted averaging unit 141 calculates a focus degree of the area of interest by performing weighted averaging on the focus degrees of the outside of the area of interest depending on the distances. The weighted averaging unit 141 calculates a focus degree fₜ by Equation (4) above.

At step S60 following step S50, the image classifier 140 classifies the intraluminal image based on the focus degree fₜ on the area of interest that is calculated by the weighted averaging unit 141 and the surface layer structure information that is calculated at step S30. As described above, the image classifier 140 classifies the intraluminal image into a focused image having a surface layer structure in the area of interest, a focused image having no surface layer structure or an unfocused image that is not focused.

As described above, according to the second embodiment of the present disclosure, an intraluminal image is classified based on surface layer structure information on an area of interest and a focus degree of the area of interest based on focus degrees of the outside of the area of interest that are calculated from a reference area that is set and distances calculated using the area of interest as a base point and thus detailed classification of a group of intraluminal images is enabled.

### Third Embodiment

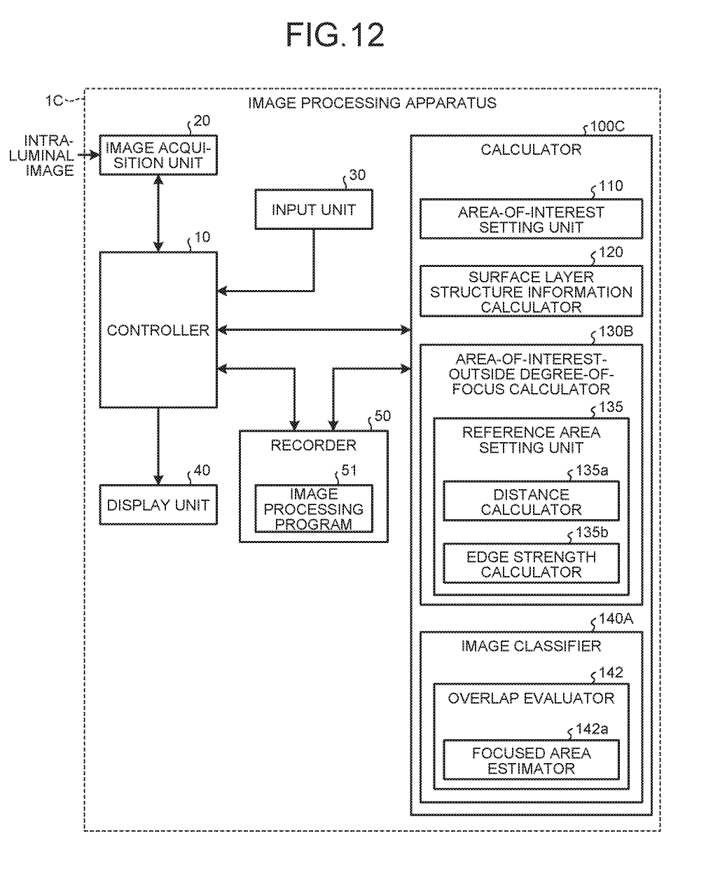

FIG. 12 is a block diagram illustrating a functional configuration of an image processing apparatus according to a third embodiment of the present disclosure. The same components as those of the image processing apparatus 1 according to the first embodiment, or the like, will be denoted with the same reference numbers as those of the first embodiment, or the like, and described. An image processing apparatus 1C illustrated in FIG. 12 includes the controller 10 that controls entire operations of the image processing apparatus 1C; the image acquisition unit 20 that acquires image data that is generated by an imaging device by capturing images of the inside of the lumen; the input unit 30 that inputs signals corresponding to external operations to the controller 10; the display unit 40 that displays various types of information and images; the recorder 50 that stores the image data that is acquired by the image acquisition unit 20 and various programs; and a calculator 100C that executes given image processing on the image data.

The calculator 100C includes the area-of-interest setting unit 110 that sets, in an acquired image, an area of interest where image classification is evaluated; the surface layer structure information calculator 120 that calculates information representing a surface layer structure in the area of interest; the area-of-interest-outside degree-of-focus calculator 130B that calculates focus degrees of on the outside of the area of interest; and an image classifier 140A that classifies the image based on the surface layer structure information and the focus degrees of the outside of the area of interest.

The image classifier 140A includes an overlap evaluator 142 that determines a focus degree of the area of interest based on a degree of overlap between a focused area and the area of interest. Furthermore, the overlap evaluator 142 includes a focused area estimator 142a that estimates a focused area from a distribution of focus degrees of the outside of the area of interest.

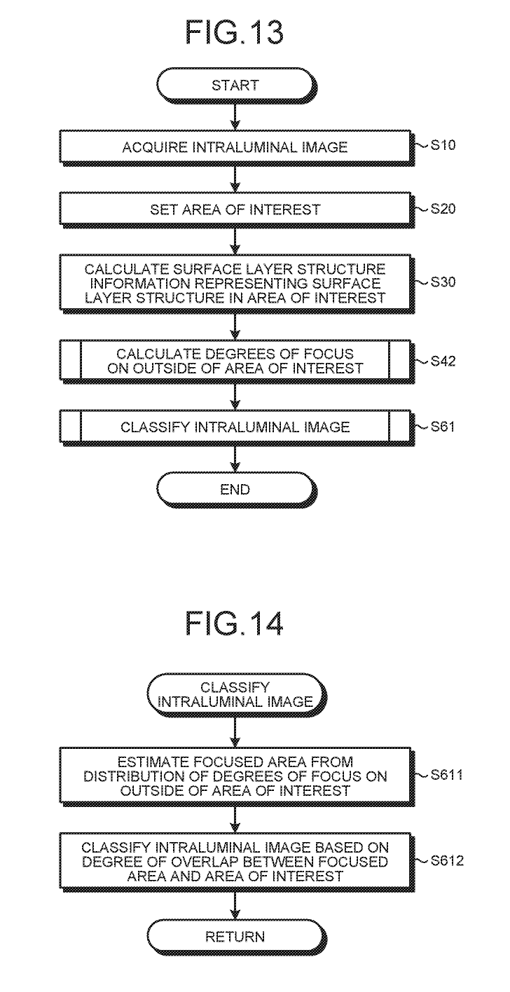

Operations of the image processing apparatus 1C will be described. FIG. 13 is a flowchart illustrating image processing performed by the image processing apparatus according to the third embodiment of the present disclosure. First of all, at step S10, the image processing apparatus 1C acquires an intraluminal image via the image acquisition unit 20.

At the following step S20, the calculator 100C sets an area of interest. As in the above-described first embodiment, the area-of-interest setting unit 110 detects a location of interest in the intraluminal image and sets an area of interest containing the location of interest.

At the following step S30, the calculator 100C calculates surface layer structure information representing a surface layer structure in the area of interest. As in the above-described first embodiment, the surface layer structure information calculator 120 calculates information representing a surface layer structure in the area of interest that is set.

At the following step S42, the calculator 100C calculates focus degrees of the outside of the area of interest. The calculator 100C calculates focus degrees of the outside of the area of interest according to the flowchart illustrated in FIG. 10.

At step S61 following step S42, the image classifier 140A classifies the intraluminal image. FIG. 14 is a flowchart illustrating a process of classifying an intraluminal image that is executed by the image classifier.

At step S611, the focused area estimator 142a estimates a focused area from the distribution of focus degrees of the outside of the area of interest. As the method of estimating a focused area, for example, a method of estimating a focused area by setting a threshold for focus degrees of the outside of the area of interest, determining sets of coordinates of focused pixels by the threshold process and performing a known closing and opening process (reference literature: CG-ARTS Association “digital image processing” revised new version, pp, 186) on the group of sets of coordinates of focused pixels can be exemplified.

At step S612, the overlap evaluator 142 determines whether the area of interest is focused based on the degree of overlap between the focused area and the area of interest. Specifically, the overlap evaluator 142 evaluates a ratio of the area in which the focused area estimated at step S611 and the area of interest overlap with each other to the area of interest. When the ratio is equal to or larger than a pre-set value, the overlap evaluator 142 determines that the area of interest is focused and, when the ratio is smaller than the pre-set value, the overlap evaluator 142 determines that the area of interest is not focused. The image classifier 140A then classifies the intraluminal image into a focused image having a surface layer structure in the area of interest, a focused image having no surface layer structure, or an unfocused image that is not focused. Thereafter, the operation of the calculator 100C returns to the main routine and the classifying process ends.

As described above, according to the third embodiment of the present disclosure, the intraluminal image is classified based on the surface layer structure information on the area of interest and the result of determining whether the area of interest is focused based on the degree of overlap between the estimated focused area and the area of interest and thus detailed classification of a group of intraluminal images is enabled. Furthermore, according to the third embodiment, the image classifier 140A is able to classify an intraluminal image without distance information and thus it is possible to improve efficiency of calculation and classify even a dark intraluminal image for which shooting distances cannot be estimated correctly.

### Fourth Embodiment

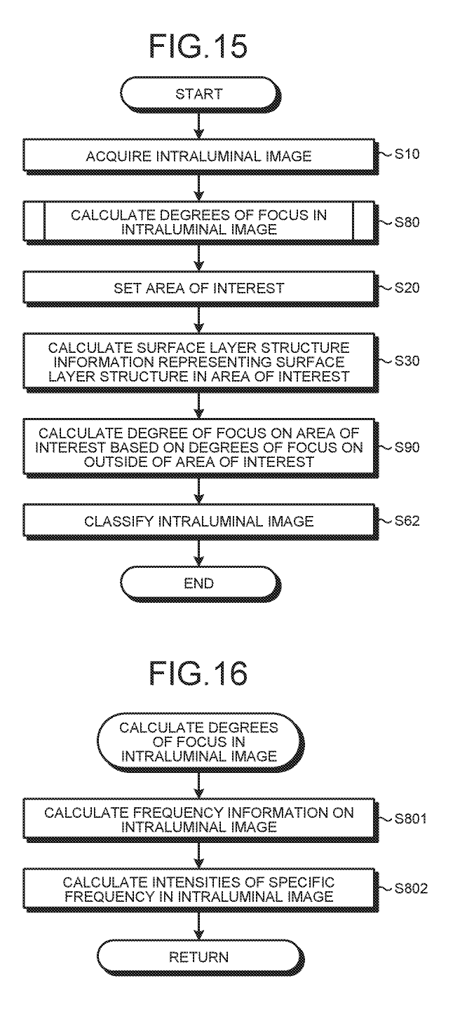

A configuration of an image processing apparatus according to a fourth embodiment is the configuration of the image processing apparatus 1 according to the above-described first embodiment excluding the distance calculator 132 and the weighted averaging unit 141. FIG. 15 is a flowchart illustrating image processing that is performed by the image processing apparatus according to the fourth embodiment of the present disclosure. First of all, at step S10, the image processing apparatus 1 acquires an intraluminal image via the image acquisition unit 20.

At step S80 following step S10, the calculator 100 calculates focus degrees in the intraluminal image. FIG. 16 is a flowchart illustrating a process of calculating focus degrees of the outside of the area of interest that is executed by the area-of-interest-outside degree-of-focus calculator.

At step S801, the frequency information calculator 131 calculates frequency information on the intraluminal image. The frequency information calculator 131 calculates frequency information on the intraluminal image in the same manner as that at step S401 in FIG. 3.

At step S802, the specific frequency intensity calculator 131a calculates intensities of a specific frequency. The specific frequency intensity calculator 131a calculates intensities of the specific frequency in the intraluminal image in the same manner as that at step S402. In this manner, at step S80, the frequency information calculator 131 calculates intensities of the specific frequency at all pixel positions in the intraluminal image as the focus degrees of at all pixel positions in the intraluminal image.

At step S20 following step S80, the calculator 100 sets an area of interest. As in the above-described first embodiment, the area-of-interest setting unit 110 detects a location of interest in the intraluminal image and sets an area of interest containing the location of interest.

At the following step S30, the calculator 100 calculates surface layer structure information representing a surface layer structure in the area of interest. As in the above-described first embodiment, the surface layer structure information calculator 120 calculates information representing a surface layer structure in the area of interest that is set.

At step S90 following step S30, the image classifier 140 calculates a focus degree of the area of interest based on the focus degrees of the outside of the area of interest. The image classifier 140 calculates a focus degree of the area of interest based on the intensities of the specific frequency outside the area of interest that are calculated at step S802. Specifically, the image classifier 140 calculates a representative value of the focus degrees of the outside of the area of interest as the focus degree of the area of interest. The weighted averaging unit 141 may be provided and the weighted averaging unit 141 may calculate a focus degree of the area of interest by performing weighted averaging on the focus degrees of the outside of the area of interest depending on the distances.

At step S62 following step S90, the image classifier 140 classifies the intraluminal image. The image classifier 140 classifies the intraluminal image based on the calculated focus degree of the area of interest and the surface layer structure information that is calculated at step S30. As described above, the image classifier 140 classifies the intraluminal image as a focused image having a surface layer structure in the area of interest, a focused image having no surface layer structure or an unfocused image that is not focused. Thereafter, the operation of the calculator 100 returns to the main routine and the classification process ends.

As described above, according to the fourth embodiment of the present disclosure, an intraluminal image is classified based on surface layer structure information on an area of interest and a focus degree of the area of interest that is calculated from focus degrees of the outside of the area of interest and thus detailed classification of a group of intraluminal images is enabled.

### Other Embodiments

Modes for carrying out the present disclosure have been described; however, the present disclosure should not be limited only by the above-described first to fourth embodiments. For example, the first to fourth embodiments have been described as ones where an intraluminal image obtained by capturing an image of the lumen in a subject; however, embodiments are not limited thereto. An image including an evaluation target of classification, such as an image that is captured by a capsule endoscope, an industrial endoscope, or a digital camera, may be classified.

As described above, an image processing apparatus, an operation method performed by an image processing apparatus, and an operation program for an image processing apparatus according to the present disclosure are useful to perform detailed classification of images.

The present disclosure produces an effect that detailed classification of images is enabled.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising:
a processor comprising hardware, the processer being configured to execute:
setting, in an image, an area of interest where classification is evaluated;
calculating surface layer structure information representing a surface layer structure in the area of interest;
calculating at least focus degrees of the outside of the area of interest in the image; and
classifying the image based on the surface layer structure information and the focus degrees of the outside of the area of interest.

2. The image processing apparatus according to claim 1, wherein the processor is configured to classify the image as any one of a focused image having the surface layer structure, a focused image without the surface layer structure and an unfocused image.

3. The image processing apparatus according to claim 1, wherein the processor is configured to calculate frequency information on the image and calculate the focus degrees of the outside of the area of interest based on the frequency information.

4. The image processing apparatus according to claim 3, wherein the processor is configured to calculate intensities of a specific frequency band of the image based on the frequency information and obtain the calculated intensities as the focus degrees of the outside of the area of interest.

5. The image processing apparatus according to claim 1, wherein the processor is configured to
calculate distances from the area of interest to coordinates of individual pixels of the outside of the area of interest,
calculate a focus degree of the area of interest by performing a weighted operation on the focus degrees of the outside of the area of interest depending on the calculated distances, and
classify the image based on the focus degree of the area of interest.

6. The image processing apparatus according to claim 5, wherein the processor is configured to calculate, as the distance,
a distance on the image between a given set of coordinates in the area of interest and the coordinates of individual pixels of the outside of the area of interest on the image, or
a difference between a shooting distance to a subject reflected in the area of interest and a shooting distance to a subject reflected in each pixel.

7. The image processing apparatus according to claim 5, wherein the processor is configured to calculate a degree of focus on the outside of the area of interest having the distance within a preset range.

8. The image processing apparatus according to claim 5, wherein the processor is configured to perform weighted averaging as the weighted operation.

9. The image processing apparatus according to claim 1, wherein the processor is configured to estimate a shooting distance for each coordinate of individual pixels in the image and calculate each focus degrees of the outside of the area of interest by using a parameter corresponding to the shooting distance.

10. The image processing apparatus according to claim 9, wherein the processor is configured to calculate frequency information that differs depending on the shooting distance and calculate the focus degrees of the outside of the area of interest based on the calculated frequency information.

11. The image processing apparatus according to claim 1, wherein the processor is configured to set a reference area outside the area of interest in the image and calculate the focus degrees of the outside of the area of interest based on information on the reference area.

12. The image processing apparatus according to claim 11, wherein the processor is configured to calculate a distance from the area of interest to coordinates of individual pixels outside the area of interest and set a reference area where only coordinates of pixels corresponding to the calculated distance is included within a preset range.

13. The image processing apparatus according to claim 12, wherein the processor is configured to calculate a strength of an edge in the image and set the reference area when the edge having an intensity equal to or larger than a given intensity does not exist between the area of interest and the reference area.

14. The image processing apparatus according to claim 1, wherein the processor is configured to
estimate a focused area from a distribution of the focus degrees of the outside of the area of interest,
evaluate a degree of overlap between the focused area and the area of interest,
determine whether the area of interest is focused based on the degree of overlap onto the area of interest, and
classify the image based on a result of the determination.

15. The image processing apparatus according to claim 1, wherein
the area of interest is an area containing a lesion, and
the processor is configured to determine a focus degree of the area of interest.

16. The image processing apparatus according to claim 1, wherein the image is an intraluminal image obtained by capturing an inside of a lumen.

17. An operation method for an image processing apparatus, the method comprising:
setting, in an image, an area of interest where classification is evaluated;
calculating surface layer structure information representing a surface layer structure in the area of interest;
calculating at least focus degrees of the outside of the area of interest in the image; and
classifying the image based on the surface layer structure information and the focus degrees of the outside of the area of interest.

18. A non-transitory computer-readable recording medium on which an executable program is recorded, the program instructing a processor of an image processing apparatus to execute:
setting, in an image, an area of interest where classification is evaluated;
calculating surface layer structure information representing a surface layer structure in the area of interest;
calculating at least focus degrees of the outside of the area of interest in the image; and
classifying the image based on the surface layer structure information and the focus degrees of the outside of the area of interest.
